(19) United States
(12) Patent Application Publication
(10) Pub. No.: US 2015/0203526 A1
(43) Pub. Date: Jul. 23, 2015

(54) NOVEL 19-NOR-STEROIDS AND THEIR USE FOR TREATING PROGESTERONE- DEPENDENT CONDITIONS

(71) Applicant: REPROS THERAPEUTICS INC., The Woodlands, TX (US)

(72) Inventors: Joseph S. Podolski, The Woodlands, TX (US); Ronald D. Wiehle, Houston, TX (US)

(73) Assignee: REPROS THERAPEUTICS INC., The Woodlands, TX (US)

(21) Appl. No.: 14/664,518

(22) Filed: Mar. 20, 2015

Related U.S. Application Data
(63) Continuation of application No. 13/997,097, filed on Jun. 21, 2013, now abandoned.
Application No. 13/997,097 is a continuation-in-part of application No. PCT/US2010/062068, filed on Dec. 23, 2010.
(60) Provisional application No. 61/316,263, filed on Mar. 22, 2010.

(51) Int. Cl. C07J 7/00

(52) U.S. Cl. CPC C07J 7/008; C07J 7/0045

(57) ABSTRACT

The subject matter of the instant invention is pertinent to the field of treatment of hormone-dependent conditions. New compounds and methods for treating these conditions are disclosed. Embodiments of the instant invention disclose methods for treating endometriosis, dysmenorrhea, breast cancer, uterine fibroids and endometrial hyperproliferation.

### CROSS-REFERENCE TO RELATED APPLICATIONS

This application is a continuation of U.S. application Ser. No. 13/997,097 filed Jun. 21, 2013, which is a U.S. National Stage of International Patent Application No. PCT/US2011/050859, filed Sep. 8, 2011, which is a continuation in part of International Application Number PCT/US2010/062068, filed Dec. 23, 2010, which claims the benefit of U.S. Application No. 61/316,263, filed Mar. 22, 2010, the contents of each which are hereby incorporated by reference.

### FIELD OF THE INVENTION

In several embodiments, the present invention relates to 19-norsteroid progesterone receptor modulators with reduced liver toxicity and improved solubility, compositions comprising same and use of these progesterone receptor modulators to treat progesterone-dependent conditions.

### BACKGROUND OF THE INVENTION

The effect of the steroid hormone progesterone on the reproductive system has been well-documented. For example, progesterone is vital to establishing and maintaining pregnancy and exerts actions on various tissues of the reproductive system. The action of progesterone on tissues outside the reproductive system has been reported but is less well characterized.

Antiprogestins, compounds which inhibit the action of progesterone, have considerable potential for use in the pharmacological regulation of fertility and a variety of conditions and diseases such as breast cancer and endometriosis. The first reported antiprogestin, mifepristone (RU 486), is one of a number of 19-nortestsosterone derivatives with strong affinity for both the progesterone and glucocorticoid receptors and with antiprogestational and antiglucocorticoid activity. A variety of antiprogestins based on the 19-norprogesterone backbone have also been synthesized.

Several drawbacks are associated with the use of known antiprogestins, rendering them less than ideal for chronic administration. If these and other limitations associated with antiprogestin treatment could be improved, a significant advance in the treatment of hormone-dependent disorders would result.

### SUMMARY OF THE INVENTION

In one embodiment, the present invention provides new steroids which possess potent antiprogestational activity, minimal antiglucocorticoid activity and reduced liver toxicity. The new steroids may also possess improved handling properties. More particularly, the present invention provides compounds having the general formula:

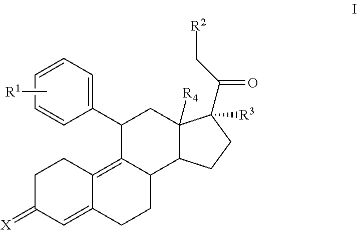

and pharmaceutically acceptable salts thereof wherein R¹, R², R³ R⁴ and X are as described below.

In a related embodiment, the present invention provides methods wherein compounds of general formula I (or pharmaceutical compositions comprising compounds of general formula I) are used to treat a variety of hormone (i.e. estrogen and/or progesterone) dependent conditions in a patient in need of such treatment. In a related embodiment, the compounds of general formula I are administered long term to treat a chronic hormone-dependent condition. In another related embodiment, the compounds of general formula I are administered by any route, including oral administration (i.e. administering to the gastrointestinal tract of a subject). In a preferred embodiment, the compounds of general formula I are administered to the vaginal mucosa for the long term treatment of a chronic hormone-dependent condition.

In another embodiment, the present invention provides methods of administering compositions comprising one or more compounds of general formula I which avoid liver toxicity.

Hormone-dependent conditions that may be treated by compositions of the invention include, without limitation, endometriosis and pain associated therewith, adenomyosis, endometriomas of the ovary, dysmenorrhea, endocrine hormone-dependent tumors, uterine fibroids, endometrial hyperproliferation, ovarian cancer, cervical cancer and breast cancer. Compositions of the instant invention may also be used to induce menses, to induce labor and for contraception.

### BRIEF DESCRIPTION OF THE DRAWINGS

FIG. 1 illustrates a comparison of the Cmax (peak serum concentration) and area under the curve (AUC) following oral and vaginal administration of CDB-4124 or CDB-4453 at a 25 mg dose in beagles.

FIG. 2 illustrates the actual Cmax observed for Proellex (CDB-4124) and its monodemethylated metabolite CDB-4453, following oral administration of CDB-4124 at 12.5 mg, 25 mg and 50 mg doses as well as the projected Cmax for 3 mg, 6 mg and 9 mg doses. FIG. 2 also illustrates the actual Cmax observed for Proellex (CDB-4124) and its monodemethylated metabolite CDB-4453, following vaginal administration of CDB-4124 at 12.5 mg, 25 mg and 50 mg doses.

FIG. 3 illustrates a comparison of the inhibition of progesterone-induced endometrial proliferation in estradiol-primed immature rabbits following subcutaneous injection and oral administration of CDB-4124

FIG. 4 compares the antiprogestational effects of three doses of CDB-4124 when delivered orally versus when delivered to the vaginal mucosa of estradiol-primed immature rabbits in the presence of progesterone, as measured by a decrease in the McPhail index. Treatment with progesterone alone (vehicle control) provided a baseline measurement of progestational activity.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

It is to be understood that any ranges, ratios and ranges of ratios that can be formed by any of the numbers or data present herein represent further embodiments of the present invention. This includes ranges that can be formed that do or do not include a finite upper and/or lower boundary. Accordingly, the skilled person will appreciate that many such ratios, ranges and ranges of ratios can be unambiguously derived form the data and numbers presented herein and all represent embodiments of the invention.

Before the present compounds, compositions and methods are disclosed and described, it is to be understood that the terminology used herein is for the purpose of describing particular embodiments only and is not intended to be limiting. It must be noted that, as used in the present specification and the appended claims, the singular forms “a,” “an” and “the” include plural referents unless the context clearly dictates otherwise.

Definitions

The term “oral” administration means that the active agent is in a formulation designed to be ingested, i.e. designed to be delivered to the gastrointestinal system for absorption.

The term “effective dosage” means an amount of the composition's active component sufficient to treat a particular condition.

The term “selective progesterone receptor modulators” means compounds that affect functions of progesterone receptor in a tissue-specific manner. The compounds act as progesterone receptor antagonists in some tissues (for example, in breast tissue) and as progesterone receptor agonists in other tissues (for example, in the uterus).

The term “treat” or “treatment” as used herein refers to any treatment of any progesterone-dependent disorder or disease, and includes, but is not limited to, inhibiting the disorder or disease arresting the development of the disorder or disease; relieving the disorder or disease, for example, causing regression of the disorder or disease; or relieving the condition caused by the disease or disorder, relieving the symptoms of the disease or disorder.

The term “prevent” or “prevention,” in relation to a progesterone-dependent disorder or disease, means preventing the onset of disorder or disease development if none had occurred, or preventing further disorder or disease development if the disorder or disease was already present. For example, compositions of the present invention may be used to prevent the recurrence of tumors. Recurrence of tumors may occur because of residual microscopic groups or nests of tumor cells which subsequently expand into clinically detectable tumors.

The term “progesterone agonist” means a compound that binds to a progesterone receptor and mimics the action of the natural hormone.

The term “progesterone antagonist” means a compound that binds to a progesterone receptor and inhibits the effect of progesterone.

The term “not substantially reduced” as used herein in reference to hormone levels in a female means that hormone levels are maintained within the normal range during administration of compositions of the invention. Thus, it is considered that some reduction in a hormone level may occur so long as the hormone level is maintained within the normal range.

The term “not substantially increased” as used herein in reference to hormone levels in a female means that hormone levels are maintained within the normal range during administration of compositions of the instant invention. Thus, it is considered that some elevation in a hormone level may occur so long as the hormone level is maintained within the normal range.

The term “alkyl” used herein refers to a straight chain, branched or cyclic saturated aliphatic hydrocarbon having from 1-12 carbons and preferably from 1-6 carbons in which case the term “lower alkyl” is descriptive. As used herein the term “alkyl” encompasses “substituted alkyls” which refers to alkyl as described including one or more functional groups such as aryl, acyl, halogen, hydroxy (e.g. hydroxymethyl), amino, acyloxy, alkoxy (e.g. methoxymethyl), and the like. These groups may be attached to any carbon atom of the alkyl moiety. The term “alkynyl”, denoting linear or branched radicals having at least one carbon-carbon triple bond is not encompassed by the term “alkyl”.

The term “alkenyl” used herein refers to a monovalent unbranched or branched hydrocarbon chain having one or more double bonds therein including without limitation C₂-C₈ alkenyl groups such as vinyl, allyl, butenyl, pentenyl, hexenyl. The term “alkenyl” embraces radicals having “cis” and “trans” orientations. The alkenyl group can be unsubstituted or substituted with one or two suitable substituents. The term “alkynyl”, denoting linear or branched radicals having at least one carbon-carbon triple bond is not encompassed by the term “alkenyl”.

The term “acyloxy” used herein refers to an organic radical derived from an organic acid by the removal of a hydrogen such as acetoxy, formyloxy, and the like. The organic radical can be further substituted with one or more functional groups such as alkyl, aryl, aralkyl, acyl, halogen, amino (e.g. glcyinate), thiol, hydroxy, alkoxy, and the like.

The term “acyl” used herein refers to groups —C(O)R, where R is alkyl or aryl (substituted or unsubstituted)

The term “alkoxy” used herein refers to the —OR group where R is a lower alkyl, aryl, or aralkyl and include without limitation methoxy, ethoxy, phenoxy, methoxyethoxy, t-butoxy and the like.

The term “hydroxy” used herein refers to the group —OH.

The term “aryl” used herein refers to an aromatic substituted which may be a single ring or multiple rings fused together, linked covalently or linked to a common group such as an ethylene or methylene moiety and include phenyl, naphthyl, biphenyl, and may contain a heteroatom such as thienyl and pryidyl. The aryl group may be substituted with halogen atoms, carboxyl, alkoxy, and the like.

Compounds

In one aspect, the present invention provides compounds having the general formula:

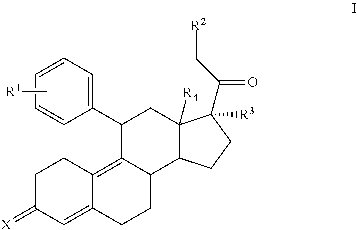

and pharmaceutically acceptable salts thereof wherein, R¹ may be at the para, ortho or meta position and is a functional group including, but not limited to, —CH(OH)CH₃; alkyl; alkenyl; cycloalkyl; cycloalkenyl; aryl; alkylsulfinyl (e.g. CH₃SO); alkylsulfonyl (e.g. CH₃SO₂); acyl (e.g. formyl, acetyl, propionyl, butyryl and the like); alkoxy (e.g. —OCH₃, —O(CH₂)₂CH₃, —O—CH₂—CH═CH₂); thioalkoxy; thioalkyl (—SCH₃), acyloxy (e.g. acetoxy, propanoyloxy); Si(CH₃)₃;

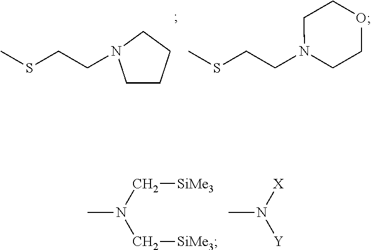

wherein X and Y are acyl; and a heterocyle preferably containing at least one nitrogen atom (e.g. aziridinyl

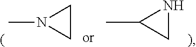

azirinyl

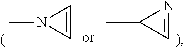

azetidinyl, pyrrolidinyl (—NC₄H₈), substituted pyrrolidinyl (e.g. methoxypyrrolidinyl, ethoxypyrrolidinyl), pyrrole

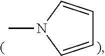

piperidinyl (—NC₅H₁₀), substituted piperidinyl (e.g. —O(CH₂)₂NC₅H₁₀), pyridinyl

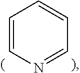

morpholinyl (NC₄H₈O), substituted morpholinyl (e.g. ethoxymorpholinyl), oxazinyl, piperazinyl

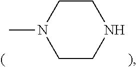

substituted piperazinyl

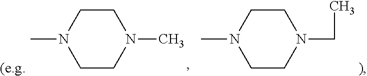

diazinyl, and an azole such as pyrazole

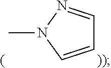

R² is a functional group including, but not limited to, hydrogen, halogen, alkyl, acyl, hydroxyl, alkoxy (e.g. methoxy, ethoxy, vinyloxy, ethynyloxy, cyclopropyloxy, etc.), acyloxy (e.g. formyloxy, acetoxy, propionyloxy, heptanoyloxy, glycinate, etc.), alkyl carbonate, cypionyloxy, S-alkyl, S—CN, S-acyl and —OC(O)R⁶ wherein R⁶ is functional group including alkyl, alkoxyalkyl (e.g. —CH₂OCH₃) or alkoxy (—OCH₃); R³ is a functional group including but not limited to alkyl (e.g. methyl, methoxymethyl), hydroxy, alkoxy (e.g. methoxy, ethoxy, methoxyethoxy, etc), and acyloxy; R⁴ is a functional group including but not limited to hydrogen and alkyl; and X is a functional group including but not limited to ═O, ═N—OR5 wherein R5 is hydrogen or alkyl, OH, CH₂, OAlk₁, and OCOAlk₂, wherein Alk₁ and Alk₂ are C1-C8 alkyl or C7-C15 aralalkyl, with the proviso that if R¹ is at the para position and is —OCH₃, —SCH₃, —NC₄H₈, —NC₅H₁₀, —NC₄H₈O, —CHO, —CH(OH)CH₃, —COCH₃, —O(CH₂)₂NC₄H₈, or —O(CH₂)₂NC₅H₁₀, X is other than ═O or ═N—OR5 wherein R5 is hydrogen or alkyl and with the proviso that if R² is hydrogen, R³ is hydroxy or methyl, R⁴ is methyl, and X is ═O, R¹ is other than methoxy, isopropyl, phenyl or hydrogen.

In one preferred embodiment, a compound of general formula I or a pharmaceutically acceptable salt thereof is provided wherein: R¹ is at the para position and is —OCH₃, —SCH₃, —NC₄H₈ (pyrrolidine), —NC₅H₁₀ (piperidino), —NC₄H₈O (morpholino), —CHO, —CH(OH)CH₃, —COCH₃, —O(CH₂)₂NC₄H₈ (methoxypyrrolidino) or —O(CH₂)₂NC₅H₁₀ (ethoxypiperidinophenyl); R² is hydrogen, halogen, alkyl, acyl, hydroxyl, alkoxy (e.g. methoxy, ethoxy, vinyloxy, ethynyloxy, cyclopropyloxy, etc.), acyloxy (e.g. formyloxy, acetoxy, propionyloxy, heptanoyloxy, glycinate, etc.), alkyl carbonate, cypionyloxy, S-alkyl, S—CN, S-acyl and —OC(O)R⁶ wherein R⁶ is functional group including alkyl, alkoxyalkyl (e.g. —CH₂OCH₃) or alkoxy (e.g. —OCH₃); R³ is alkyl (e.g. methyl, methoxymethyl), hydroxy, alkoxy (e.g. methoxy, ethoxy, methoxyethoxy, etc), or acyloxy; R⁴ is hydrogen or alkyl; and X is OH, CH₂, OAlk1, or OCOAlk2, wherein Alk1 and Alk2 are C1-C8 alkyl or C7-C15 aralalkyl. In a particularly preferred embodiment, R¹ is at the para position and is —COCH₃ or —CHO, R² is alkoxy, R³ is alkyl, hydroxy, alkoxy or acyloxy, R4 is alkyl and X is OH, CH₂, OAlk1, or OCOAlk2, wherein Alk1 and Alk2 are C1-C8 alkyl or C7-C15 aralalkyl. Even more preferably, R¹ is at the para position and is —COCH₃, R² is methoxy, R³ is acetoxy, R4 is methyl, and X is OH, CH₂, OAlk1, or OCOAlk2, wherein Alk1 and Alk2 are C1-C8 alkyl or C7-C15 aralalkyl.

In another preferred embodiment, a compound of general formula I or a pharmaceutically acceptable salt thereof is provided wherein R¹ is at the meta or ortho position and is —OCH₃, —SCH₃, —NC₄H₈ (pyrrolidine), —NC₅H₁₀ (piperidino), —NC₄H₈O (morpholino), —CHO, —CH(OH)CH₃, —COCH₃, —O(CH₂)₂NC₄H₈ (methoxypyrrolidino) or —O(CH₂)₂NC₅H₁₀ (ethoxypiperidinophenyl); R² is hydrogen, halogen, alkyl, acyl, hydroxyl, alkoxy (e.g. methoxy, ethoxy, vinyloxy, ethynyloxy, cyclopropyloxy, etc.), acyloxy (e.g. formyloxy, acetoxy, propionyloxy, heptanoyloxy, glycinate, etc.), alkyl carbonate, cypionyloxy, S-alkyl, S—CN, S-acyl and —OC(O)R⁶ wherein R⁶ is functional group including alkyl, alkoxyalkyl (e.g. —CH₂OCH₃) or alkoxy (—OCH₃); R³ is alkyl (e.g. methyl, methoxymethyl), hydroxy, alkoxy (e.g. methoxy, ethoxy, methoxyethoxy, etc), or acyloxy; R⁴ is hydrogen or alkyl; and X is ═O, ═N—OR5 wherein R5 is hydrogen or alkyl, OH, CH₂, OAlk1, or OCOAlk2, wherein Alk1 and Alk2 are C1-C8 alkyl or C7-C15 aralalkyl with the proviso that if R² is hydrogen, R³ is hydroxy, R⁴ is methyl, and X is ═O, R¹ is other than methoxy. In a particularly preferred embodiment, R¹ is at the meta or ortho position and is —COCH₃ or —CHO, R² is alkoxy, acyloxy or hydrogen, R³ is alkyl, hydroxy, alkoxy or acyloxy, R4 is alkyl and X is ═O, ═N—OR5 wherein R5 is hydrogen or alkyl, OH, CH₂, OAlk1, or OCOAlk2, wherein Alk1 and Alk2 are C1-C8 alkyl or C7-C15 aralalkyl. Particularly preferred compounds include 21-methoxy-17α-acetoxy-11β-(3-acetylphenyl)-19-norpregna-4,9-diene-3,20-dione (in which R¹ is at the meta position and is —COCH₃, R² is methoxy, R³ is acetoxy, R4 is methyl, and X is ═O) with the following structural formula:

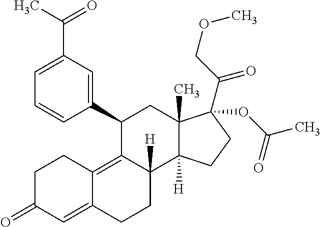

17α-acetoxy-11β-(3-acetylphenyl)-19-norpregna-4,9-dien-3,20-dione (in which R¹ is at the meta position and is —COCH₃, R² is hydrogen, R³ is acetoxy, R4 is methyl, and X is ═O) with the following structural formula:

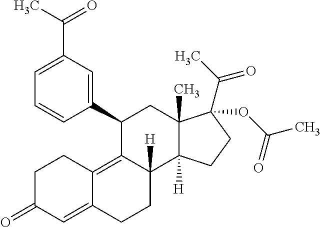

and 21-acetoxy-17α-acetoxy-11β-(3-acetylphenyl)-19-norpregna-4,9-diene-3,20-dione (in which R¹ is at the meta position and is —COCH₃, R² and R³ are acetoxy, R4 is methyl, and X is ═O) with the following structural formula:

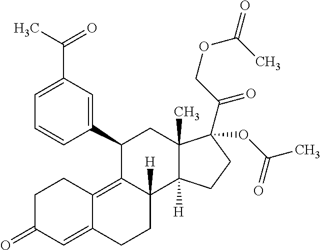

In yet another preferred embodiment, a compound of general formula I or a pharmaceutically acceptable salt thereof is provided wherein R¹ is at the para position and is alkyl; alkenyl; cycloalkyl; cycloalkenyl; aryl; alkylsulfinyl (e.g. methylsulfinyl); alkylsulfonyl (e.g. SO2CH3); thioalkoxy; Si(CH₃)₃;

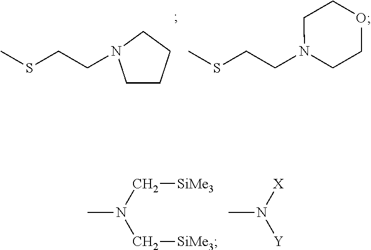

wherein X and Y are acyl; aziridinyl, azirinyl, azetidinyl, methoxypyrrolidinyl, ethoxymorpholinyl, oxazinyl, piperazinyl, methylpiperazinyl, ethylpiperazinyl or diazinyl; R² is hydrogen, halogen, alkyl, acyl, hydroxyl, alkoxy (e.g. methoxy, ethoxy, vinyloxy, ethynyloxy, cyclopropyloxy, etc.), acyloxy (e.g. formyloxy, acetoxy, propionyloxy, heptanoyloxy, glycinate, etc.), alkyl carbonate, cypionyloxy, S-alkyl, S—CN, S-acyl or —OC(O)R⁶ wherein R⁶ is functional group including alkyl, alkoxyalkyl (e.g. —CH₂OCH₃) or alkoxy (—OCH₃); R³ is alkyl (e.g. methyl, methoxymethyl), hydroxy, alkoxy (e.g. methoxy, ethoxy, methoxyethoxy, etc), or acyloxy; R⁴ is hydrogen or alkyl; and X is ═O, ═N—OR5 wherein R5 is hydrogen or alkyl, OH, CH₂, OAlk1, or OCOAlk2, wherein Alk1 and Alk2 are C1-C8 alkyl or C7-C15 aralalkyl “with the proviso that if R² is hydrogen, R³ is hydroxy or methyl, R⁴ is methyl, and X is ═O, R¹ is other than isopropyl or phenyl. In a particularly preferred embodiment, R¹ is at the para position and is alkylsulfinyl, R² is alkoxy, R³ is alkyl, hydroxy, alkoxy or acyloxy, R4 is alkyl and X is ═O, ═N—OR5 wherein R5 is hydrogen or alkyl, OH, CH₂, OAlk1, or OCOAlk2, wherein Alk1 and Alk2 are C1-C8 alkyl or C7-C15 aralalkyl. Even more preferably, R¹ is at the para position and is —SOCH₃, R² is methoxy, R³ is acetoxy, R⁴ is methyl, and X is ═O.

In yet another preferred embodiment, a compound of general formula I or a pharmaceutically acceptable salt thereof is provided wherein R¹ is at the meta or ortho position and is alkyl; alkenyl; cycloalkyl; cycloalkenyl; aryl; alkylsulfinyl (e.g. CH₃SO); alkylsulfonyl (e.g. CH₃SO₂); thioalkoxy; Si(CH₃)₃;

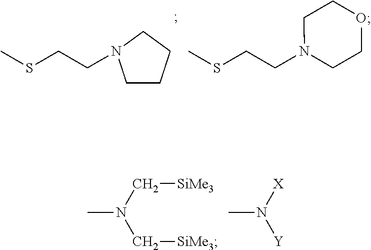

wherein X and Y are acyl; aziridinyl, azirinyl, azetidinyl, methoxypyrrolidinyl, ethoxymorpholinyl, oxazinyl, piperazinyl, methylpiperazinyl, ethylpiperazinyl or diazinyl; R² is hydrogen, halogen, alkyl, acyl, hydroxyl, alkoxy (e.g. methoxy, ethoxy, vinyloxy, ethynyloxy, cyclopropyloxy, etc.), acyloxy (e.g. formyloxy, acetoxy, propionyloxy, heptanoyloxy, glycinate, etc.), alkyl carbonate, cypionyloxy, S-alkyl, S—CN, S-acyl or —OC(O)R⁶ wherein R⁶ is functional group including alkyl, alkoxyalkyl (e.g. —CH₂OCH₃) or alkoxy (—OCH₃); R³ is alkyl (e.g. methyl, methoxymethyl), hydroxy, alkoxy (e.g. methoxy, ethoxy, methoxyethoxy, etc), or acyloxy; R⁴ is hydrogen or alkyl; and X is ═O, ═N—OR5 wherein R5 is hydrogen or alkyl, OH, CH₂, OAlk1, or OCOAlk2, wherein Alk1 and Alk2 are C1-C8 alkyl or C7-C15 aralalkyl. In a particularly preferred embodiment, R¹ is at the ortho or meta position and is alkylsulfinyl, R² is alkoxy, R³ is alkyl, hydroxy, alkoxy or acyloxy, R4 is alkyl and X is ═O, ═N—OR5 wherein R5 is hydrogen or alkyl, OH, CH₂, OAlk1, or OCOAlk2, wherein Alk1 and Alk2 are C1-C8 alkyl or C7-C15 aralalkyl. Even more preferably, R¹ is at the meta position and is —SOCH₃, R² is methoxy, R³ is acetoxy, R4 is methyl, and X is ═O.

Particularly preferred R¹ substituents, whether at the ortho, meta or para position, are —CHO, —COCH₃ and —SOCH₃.

Particularly preferred R² substituents are alkoxy (particularly methoxy or ethoxy) and hydrogen.

Particularly preferred R³ substituents are alkoxy (particularly methoxy or ethoxy) and acyloxy (particularly acetoxy, propionyloxy, and formyloxy).

Particularly preferred R⁴ substituents are alkyls, preferably methyl.

A particularly preferred X substituent is ═O.

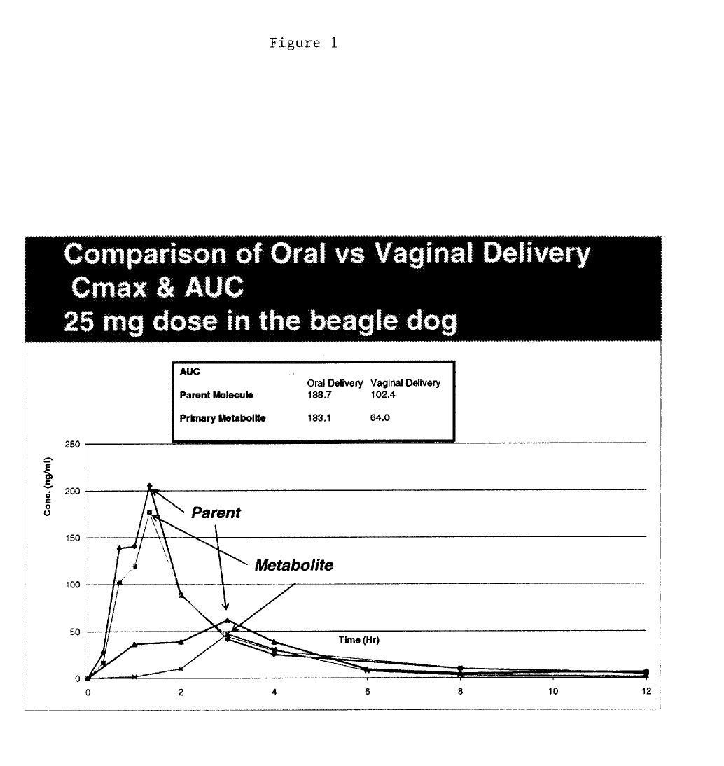

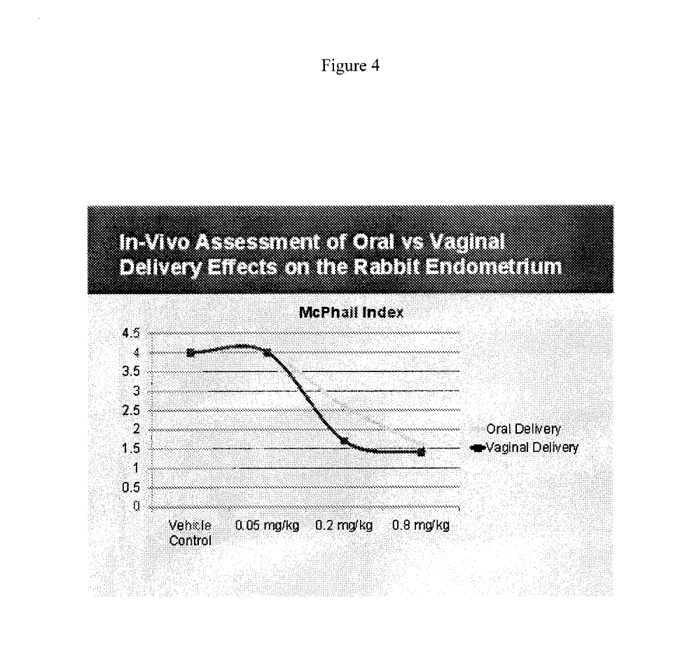

The compounds of general formula I can be synthesized by conventional synthetic chemistry techniques, including those techniques used to synthesize the compounds disclosed in U.S. Pat. Nos. 6,861,415, 6,900,193, and 6,020,328, the contents of each of which are hereby incorporated by reference. In particular, the synthetic schemes set forth in FIGS. 1, 2 and 3 of U.S. Pat. No. 6,861,415 and FIGS. 1-11 of U.S. Pat. No. may be used, in combination with synthetic techniques known in the art such as those described in 6,020,328, to synthesize compounds of the present invention.

Compounds of general formula I possess a phenyl group at C11β, which is substituted at the ortho, meta or para position (i.e. at position R¹ of general formula I) with a functional group that cannot be metabolized to produce a primary amine upon administration of the compound. For example, compounds having a dimethylaminophenyl group at the C11β position undergo dealkylation upon administration to yield the primary amine aniline(-phenyl-NH₂) at the C11β position. The dealkylation occurs in two steps: first, the dimethylaminophenyl group is monodemethylated relatively quickly to monomethylaminophenyl; second, in a relatively slow reaction, the remaining alkyl group is removed to form the primary amine. Without being bound by theory, it is believed that aniline or substituted aniline (phenyl-NRH) groups may serve as reactive nucleophiles contributing to adverse liver reactions in patients who have received these compounds by the formation of protein adducts, particularly when administered long term at relatively high doses. Accordingly, R¹ is not a primary, secondary or tertiary amine. Moreover, R¹ is not a functional group other than a primary, secondary or tertiary amine which is itself substituted with a primary, secondary or tertiary amine. The compounds of the present invention are therefore unexpectedly useful for the long-term treatment of hormone-dependent disorders.

Certain compounds of general formula I may also have improved solubility in various solvents including aqueous and alcohol (e.g. ethanol)-based solvents. In particular, the present inventors have discovered that compounds of general formula I having R¹=acyl (particularly COCH₃), alkylsulfinyl or alkylsulfonyl at the meta (or ortho) position and having R²=alkoxy, particularly methoxy, may be surprisingly soluble in a variety of polar solvents (i.e. having a dielectric constant of at least 15), possess potent antiprogestational activity and minimal antiglucocorticoid activity, making them particularly suitable as therapeutic agents for the treatment of progesterone-dependent disorders.

In a related embodiment, the present invention relates to methods of treating a progesterone-dependent condition by administering one or more compounds of general Formula I (or a pharmaceutical composition comprising one or more compounds of general Formula I) as described above. Compounds of general Formula I are not expected to contribute to adverse liver reactions in patients who have received these compounds and therefore, according to this aspect of the present invention, may be administered through any route, including without limitation oral (i.e. administration to the gastrointestinal tract), sublingual/buccal, intravascular, intramuscular, subcutaneous, inhalation, mucosal (e.g. rectal or vaginal), and topical. In a preferred embodiment, a composition comprising one or more compounds of general Formula I is administered orally at a dosage of at least 25 mg/day, more preferably at least 50 mg/day, to treat a hormone-dependent condition for a period of at least 2, 3, 4, 5, 6, 7, 8, 9, 10 or more months.

Methods

Also provided by the present invention are methods of administering antiprogestins for the treatment of hormone (e.g. progesterone) dependent conditions which avoid liver toxicity.

In one embodiment, the present invention relates to methods of treating a progesterone-dependent condition by oral administration of a compound of general formula I, preferably at a dosage of at least 25 mg/day, more preferably at least 50 mg/day. Compounds of general formula I may be orally administered daily (i.e. at least once per day for a consecutive period of days) for an administration period which may continue for at least 2, 3, 4, 5, 6, 7, 8, 9, 10 or more months.

In another embodiment, the present invention relates to non-oral administration of a composition comprising one or more compounds of general formula I to treat a hormone (e.g. progesterone) dependent condition. This aspect of the invention arises in part from the unexpected finding that certain 19-nortestosterone- or 19-norprogesterone-derived antiprogestins can exhibit toxic effects on the liver at therapeutic concentrations, limiting their clinical use. Specifically, it has been found that patients subjected to chronic daily administration of therapeutic oral (i.e., for ingestion) dosages of the antiprogestin/SPRM CDB-4124 exhibit liver toxicity. Large amounts of the mono-demethylated metabolite of CDB-4124 are detected by pharmacokinetic studies on patients subsequent to oral ingestion of CDB-4124, indicating CDB-4124 undergoes significant first pass metabolism in the liver providing the opportunity for liver damage. Compounds of formula I have C11β substituents which are not expected to form protein adducts in the liver and toxic liver effects are further avoided by circumventing first-pass metabolism by administering the compounds non-orally.

In a related embodiment, the compounds are administered non-orally at a therapeutically effective dose that is relatively low compared to the therapeutically effective dose of the compound when administered orally. For example, when administered locally to the vaginal mucosa, the therapeutically effective dose may be less than 50 mg/day, less than 40 mg/day, less than 30 mg/day less than 20 mg/day, less than 10 mg/day, less than 5 mg/day, between 5 mg/day and 50 mg/day, between 5 mg/day and 40 mg/day, between 5 mg/day and 30 mg/day, between 5 mg/day and 20 mg/day, or between 5 mg/day and 10 mg/day. In another related embodiment, the effective amount of the compound is less than the effective amount when administered systemically, for example, the effective amount when administered locally to the vaginal mucosa may be 2-fold, 3-fold, 4-fold 5-fold, 6-fold, 7-fold, 8-fold, 9-fold and even 10-fold less than the effective amount when administered systemically to treat endometriosis, uterine fibroids and other diseases located in that region.

Compounds of general Formula I as described above are expected to exhibit reduced or no liver toxicity whether delivered through an oral or non-oral route, making them suitable for use in treating various progesterone-dependent conditions when administered via any administration route including without limitation oral, sublingual/buccal, intravascular, intramuscular, subcutaneous, inhalation, mucosal (e.g. rectal or vaginal), and topical.

Non-oral administration of compounds of general Formula I may reduce liver toxicity (if present) compared to oral administration of the same compounds. When administered non-orally, the compounds are preferably administered by a route which avoids first pass metabolism such as, without limitation, intravenous, intramuscular, sublingual and mucusoal (e.g. vaginal, intrauterine or rectal).

In one embodiment of the invention, a composition of the invention is administered to a patient with breast cancer in order to treat the breast cancer. In a preferred embodiment, the patient is a human female and the breast cancer expresses human estrogen receptor (hER) or human progesterone receptor (hPR) and more preferably expresses both hER and hPR.

In a related embodiment of the invention, a composition of the invention is administered to a breast cancer patient with one or more tumors resistant to antiestrogen treatments in order to treat the breast cancer. For example, compounds of the instant invention may be particularly useful for treating tamoxifen-resistant breast cancer in patients.

In a related embodiment of the invention, a composition of the invention is administered to a patient suffering from a disorder selected from the group consisting of ductal carcinoma in situ (DCIS), muscinous (colloid) carcinoma, medullary carcinoma of the breast, papillary carcinoma of the breast, adenoid cystic carcinoma (ACC), Paget's disease of the nipple, inflammatory breast disease, fibroadenoma and fibrocystic breast disease in order to treat the disorder.

In another embodiment of the invention, a composition of the instant invention is administered to a female undergoing estrogen therapy in order to prevent the development of breast cancer in the female.

In a related embodiment, the composition is administered by a (non-oral) route that avoids first pass metabolism selected from the group consisting of: sublingual/buccal, intravascular, intramuscular, subcutaneous, inhalation, mucosal (e.g. rectal, intrauterine or vaginal), and topical. In a preferred embodiment, a composition of the invention is administered to a breast cancer patient in the form of a trans-dermal patch, gel or ointment that is applied directly to the breast (e.g. to the nipple or areola) in order to treat the breast cancer.

In another embodiment of the invention, a composition of the invention is administered to a female patient in need thereof in order to suppress endometrial proliferation. In a preferred embodiment, a composition of the invention is vaginally administered to a patient in order to suppress endometrial proliferation.

In a related embodiment of the invention, a composition of the invention is administered to a female patient in need thereof in order to treat endometriosis. In a preferred embodiment, a composition of the invention is vaginally administered to a patient in order to treat endometriosis.

In another embodiment of the invention, a composition of the invention is administered to a female in need thereof in order to treat dysmenorrhea. In a preferred embodiment, a composition of the invention is vaginally administered to a patient in order to treat dysmenorrhea.

In yet another embodiment of the invention, a composition of the invention is administered to a female in need thereof in order to treat uterine fibroids. In a preferred embodiment, a composition of the invention is vaginally administered to a patient in order to treat uterine fibroids.

In another embodiment of the invention, a composition of the invention is administered to a female patient in need thereof in order to treat adenomyosis. In a preferred embodiment, a composition of the invention is vaginally administered to a patient in order to treat adenomyosis.

In another embodiment of the invention, a composition of the invention is administered to a female patient in need thereof in order to treat an endometrioma. In a preferred embodiment, a composition of the invention is vaginally administered to a patient in order to treat an endometrioma.

In another embodiment of the invention, a composition of the invention is administered to a female patient in need thereof in order to treat ovarian cancer. In a preferred embodiment, a composition of the invention is vaginally administered to a patient in order to treat ovarian cancer.

In another embodiment of the invention, a composition of the invention is administered to a female patient in need thereof in order to treat cervical cancer. In a preferred embodiment, a composition of the invention is vaginally administered to a patient in order to treat cervical cancer.

In a particularly preferred embodiment, a composition of the invention is administered to a patient suffering from endometriosis, dysmennorrhea, uterine fibroids, adenomyosis, ovarian cancer or cervical cancer by a non-oral administration route designed to provide local delivery of the compound to the affected region. The compound may be formulated into a suitable preparation for such non-oral local administration. For example, the compound may be formulated, without limitation, as a depot injection (e.g. solid or oil-based subcutaneous or intramuscular) designed to slowly release the compound over a long period of time; an intravaginal preparation such as a doughnut-shaped hormone-releasing vaginal ring; a vaginal suppository; a vaginal pill; an intra-uterine preparation such as an intrauterine device (IUD) or matrix preparation; an implantable drug delivery device; a topical gel; or a trans-dermal patch. Preferably, the compound is incorporated into a vaginal ring, uterine depot, vaginal suppository or the like which maintains a slow but continual release of the compound that is locally but not systemically significant.

In a preferred embodiment, endometriosis, dysmennorhea, uterine fibroids, adenomyosis, ovarian cancer or cervical cancer is treated by administering an intravaginal preparation containing a compound of general formula I to the vagina of a patient in need of such treating. It is understood that the compound is absorbed from the vaginal mucosa that is in direct contact with the intravaginal preparation. An intravaginal ring is a preferred intravaginal preparation and can be designed to provide continuous release of the compound in the vagina. The insertion period may be, e.g. from 1 to 3 months after which the preparation may be replaced by a new preparation to provide a continuous long term treatment.

In another preferred embodiment, endometriosis, dysmennorhea, uterine fibroids, adenomyosis, ovarian cancer or cervical cancer is treated by administering a vaginal pill or vaginal suppository containing a compound of general formula I to the vagina of a patient in need of such treating. The vaginal pill and vaginal suppository can be produced by well known methods using additives such as a diluting agent, a binding agent and a suppository base that are commonly used in the production of such preparations.

In another preferred embodiment, endometriosis, dysmennorhea, uterine fibroids, adenomyosis, ovarian cancer or cervical cancer is treated by administering an intrauterine preparation containing a compound of general formula I to the uterine cavity of a patient in need of such treating. The intrauterine preparation may be a matrix preparation which provides continuous release of the compound in the uterus. The insertion period of the intrauterine preparation may be about 6 months, after which the preparation may be removed and a new preparation inserted so that long term treatment of the disorder is achieved. The intrauterine preparation may be produced by routine methods using a matrix base (e.g. a polymer including but not limited to a silicon rubber, ethylene vinyl acetate, ethyl cellulose, carboxymethylethylcellulose, polyethylene glycol, polyvinyl alcohol, carboxyvinyl polymer or collagen) an inert intrauterine device and optionally an appropriate crosslinking agent and/or release promoting agent such as polysorbate 60, polysorbate 80, glycerin, isopropyl palmitate and isopropyl myristate. The matrix preparation may be single-layered or two-layered. The form of the intrauterine preparation is not limited but is sufficient to have suitable form for topical administration in the uterus.

In another embodiment of the invention, a composition of the invention is administered to female in need thereof in order to induce menses in the female.

In yet another embodiment of the invention, a composition of the invention is administered to a female in need thereof in order to induce labor.

In yet another embodiment of the invention, a composition of the invention is administered to female in need thereof as a contraceptive.

Compositions comprising a compound of General Formula I, as described above, may be suitable for prolonged oral administration because these compounds are expected to exhibit reduced or no liver toxicity. Alternately, a compound of General Formula I may be chronically administered by a route that avoids first pass metabolism and therefore reduces or eliminates metabolism by the liver. Thus, compositions of the invention may be administered on a chronic basis without causing toxic liver effects. Preferably, the compounds have only low glucocorticoid receptor binding activity and therefore do not interfere with functions of glucocorticoid receptor. Thus, compositions of the invention may also be associated with reduced side effects such as mood swings, fatigue and weight loss, typically found when antiprogestins with a high affinity for glucocorticoid receptor are used. Preferably, compounds of the instant invention also have low, or substantially no, estrogenic, anti-estrogenic and anti-androgenic activities.

In one embodiment, a composition of the invention comprising a compound of general formula I in an amount effective for treating a hormone dependent condition is administered for an administration period of least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or more days. The composition may also be administered for an administration period of least 1, 2, 3, 4, 5, 6, 7, 8, 9, 10, 11, 12 or more months. The composition may also be administered for an administration period of at least 1, 2, 3, 4, 5, 6, 7, 8, 9, 10 or more years. During the administration period, the composition may be administered daily or periodically such as every other day, every other month, and the like. The composition may also be administered intermittently. For example, the composition may be administered for an administration period of 1, 2, 3, 4, 5 or more months, followed by a period of discontinuance, followed by an administration period of 1, 2, 3, 4, 5 or more months, and so on.

In one embodiment, the composition is administered intermittently such that the subject undergoes menses during at least one discontinuance period. This approach is expected to avoid the adverse effects associated with a thickened or stagnant endometrium that may accompany extended treatment with progesterone antagonists, such as spotting, breakthrough bleeding, endometrial hyperproliferation or endometrial cancer. At least one, and preferably every discontinuance period is of sufficient length for the subject to experience menstruation. More preferably, the subject experiences menstruation during every discontinuance period. In a particularly preferred embodiment, the composition is administered daily for an administration period of four months, followed by a discontinuance period during which the subject experiences menstruation, followed by another administration period of four months and so on.

In one embodiment, compositions of the invention comprise a pharmaceutically acceptable salt of a compound of general formula I as described above. Depending on the process conditions the salt compound obtained may be either in neutral or salt form. Salt forms include hydrates and other solvates and also crystalline polymorphs. Both the free base and the salts of these end products may be used in accordance with the invention.

Acid addition salts may in a manner known per se be transformed into the free base using basic agents such as alkali or by ion exchange. The free base obtained may also form salts with organic or inorganic acids.

In the preparation of acid addition salts, preferably such acids are used which form suitably pharmaceutically acceptable salts. Examples of such acids are hydrochloric acid, sulfuric acid, phosphoric acid, nitric acid, aliphatic acid, alicyclic carboxylic or sulfonic acids, such as formic acid, acetic acid, propionic acid, succinic acid, glycolic acid, lactic acid, malic acid, tartaric acid, citric acid, ascorbic acid, glucuronic acid, fumaric acid, maleic acid, hydroxymaleic acid, pyruvic acid, aspartic acid, glutamic acid, p-hydroxybenzoic acid, embonic acid, ethanesulfonic acid, hydroxyethanesulfonic acid, phenylacetic acid, mandelic acid, alogenbensenesulfonic acid, toluenesulfonic acid, galactaric acid, galacturonic acid or naphthalenesulfonic acid. All crystalline form polymorphs may be used in accordance with the invention.

Base addition salts may also be used in accordance with the invention and may be prepared by contacting the free acid form with a sufficient amount of the desired base to produce the salt in the conventional manner. The free acid form may be regenerated by contacting the salt form with an acid and isolating the free acid in the conventional manner. Pharmaceutically acceptable base addition salts are formed with metals or amines, such as alkali and alkali earth metals or organic amines. Examples of metals used as cations are sodium, potassium, calcium, magnesium and the like. Examples of suitable amines are amino acids such as lysine, choline, diethanolamine, ethylenediamine, N-methylglucamine and the like.

Compositions of the instant invention can be prepared in the form of a dose unit or dose units suitable for oral, sublingual/buccal, parenteral, transdermal, transmucosal (e.g. vaginal or rectal), or topical administration. Parenteral administration includes, but is not limited to, intravenous, intraarterial, intraperitoneal, subcutaneous, intramuscular, intrathecal, and intraarticular.

In still another embodiment, compositions of the present invention are formulated as rectal suppositories, which may contain suppository bases including, but not limited to, cocoa butter or glycerides.

In still another embodiment, compositions of the present invention comprise a compound of general formula I and a bioadhesive carrier such as those described in U.S. Pat. No. 4,615,697, which is incorporated herein by reference. The bioadhesive carrier may be in gel, cream, tablet, pill, capsule, suppository, or film form or any other pharmaceutically acceptable form that will adhere to the vaginal mucosa.

Compositions of the present invention may also be formulated for inhalation, which may be in a form including, but not limited to, a solution, suspension, or emulsion that may be administered as a dry powder or in the form of an aerosol using a propellant, such as dichlorofuoromethane or trichlorofluoromethane.

Compositions of the present invention may also be formulated for transdermal delivery, for example as a cream, ointment, lotion, paste, gel, medicated plaster, patch, or membrane. Such compositions can comprise any suitable excipients, for example penetration enhancers and the like.

Compositions of the present invention may also be formulated for parenteral administration including, but not limited to, by injection or continuous infusion. Formulations for injection may be in the form of suspensions, solutions, or emulsions in oily or aqueous vehicles. Such compositions may also be provided in powder form for reconstitution with a suitable vehicle including, but not limited to, sterile, pyrogen-free water, WFI, and the like.

Compositions of the present invention may also be formulated as a depot preparation, which may be administered by implantation or by intramuscular injection. Such compositions may be formulated with suitable polymeric or hydrophobic materials (as an emulsion in an acceptable oil, for example), ion exchange resins, or as sparingly soluble derivatives (as a sparingly soluble salt, for example).

Compositions of the present invention may also be formulated as a liposome preparation. Liposome preparations can comprise liposomes which penetrate the cells of interest or the stratum corneum and fuse with the cell membrane resulting in delivery of the contents of the liposome into the cell. For example, liposomes such as those described in U.S. Pat. No. 5,077,211 to Yarosh, U.S. Pat. No. 4,621,023 to Redziniak et al., or U.S. Pat. No. 4,508,703 to Redziniak et al. can be used.

A composition of the invention can be in the form of solid dosage units such as tablets, (e.g. suspension tablets, bite suspension tablets, rapid dispersion tablets, chewable tablets, effervescent tablets, bilayer tablets, etc.), caplets, capsules (e.g., a soft or a hard gelatin capsule), powder (e.g. a packaged powder, a dispensable powder or an effervescent powder), lozenges, sachets, cachets, troches, pellets, granules, microgranules, encapsulated microgranules, powder aerosol formulations, or any other solid dosage form reasonably adapted for administration.

Suitable liquid dosage forms of a composition of the invention include solutions, aqueous or oily suspensions, elixirs, syrups, emulsions, liquid aerosol formulations, gels, creams, ointments, etc. Such compositions may also be formulated as a dry product for constitution with water or other suitable vehicle before use.

In one embodiment, liquid or semi-solid compositions, upon storage in a closed container maintained at either room temperature, refrigerated (e.g. about 5-10° C.) temperature, or freezing temperature for a period of about 1, 2, 3, 4, 5, 6, 7, 8, 9, 10, 11, or 12 months, exhibit at least about 90%, at least about 92.5%, at least about 95%, or at least about 97.5% of the original antiprogestin compound present therein.

Compositions of the invention can, if desired, include one or more pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition. Excipients include, by way of illustration and not limitation, diluents, disintegrants, binding agents, adhesives (e.g. bioadhesives), wetting agents, lubricants, glidants, surface modifying agents or surfactants, fragrances, suspending agents, emulsifying agents, nonaqueous vehicles, preservatives, antioxidants, adhesives, agents to adjust pH and osmolarity (e.g. buffering agents), preservatives, thickening agents, sweetening agents, flavoring agents, taste masking agents, colorants or dyes, penetration enhancers and substances added to improve appearance of the composition.

Compositions of the present invention may be administered in any manner including, but not limited to, orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, intraarticular, intracisternal and intraventricular.

A therapeutically effective amount of the composition required for use in therapy varies with the length of time that activity is desired, and the age and the condition of the patient to be treated, among other factors, and is ultimately determined by the attendant physician. In general, however, doses employed for human treatment typically are in the range of about 0.001 mg/kg to about 500 mg/kg per day, for example about 1 μg/kg to about 1 mg/kg per day or about 1 μg/kg to about 100 μg/kg per day. For most large mammals, the total daily dosage is from about 1 to 100 mg, preferably from about 2 to 80 mg. The dosage regimen may be adjusted to provide the optimal therapeutic response. The desired dose may be conveniently administered in a single dose, or as multiple doses administered at appropriate intervals, for example as two, three, four or more subdoses per day.

Illustratively, a composition of the invention may be administered to a subject to provide the subject with an antiprogestin in an amount of about 1 μg/kg to about 1 mg/kg body weight, for example about 1 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg or about 1 mg/kg body weight.

Patients undergoing treatments with the compositions of the instant invention should be monitored routinely for their serum estrogen and glucocorticoid levels.

The following non-limiting examples are provided to aid in understanding the teachings of the instant invention.

### EXAMPLE 1

### Measuring in vitro Binding Affinities of Antiprogestins

Competitive binding assays are performed using cytosolic preparations.

For measuring binding to rabbit progesterone receptor (PR) and glucocorticoid receptor (GR), cytosol is prepared from uterus or thymus, respectively, of estradiol-primed immature rabbits. For binding to rabbit uterine PR, cytosol containing rabbit uterine PR is prepared in TEGMD buffer (10 mM Tris, pH 7.2, 1.5 mM EDTA, 0.2 mM sodium molybdate, 10% glycerol, 1 mM DTT) and incubated with 6 nM 1,2-[³H]progesterone (NEN Life Science Products; 52 Ci/mmol); test compounds are added at concentrations from 2 to 100 nM. For binding to rabbit thymic GR, cytosol is prepared in TEGMD buffer and incubated with 6 nM 6,7-[³H]dex (NEN; 35 or 40 Ci/mmol); test compounds are added at concentrations from 2 to 100 nM.

For measuring binding to human progesterone receptor-A (rhPR-A) or progesterone receptor-B (rhPR-B), cytosolic extracts from Sf9 insect cells infected with recombinant baculovirus expressing either hPR-A or hPR-B is prepared. Sf9 cytosol (prepared in TEGMD buffer containing the following protease inhibitors: bacitracin at 100 μg/ml, aprotinin at 2 μg/ml, leupeptin at 94 μg/ml, pepstatin A at 200 μg/ml) is incubated with 6.8 nM 1,2,6,7,16,17-[³H]progesterone (NEN; 143 Ci/mmol); test compounds are added at concentrations from 1 to 100 nM.

After overnight incubation at 4 C, bound and unbound [³H]-steroids are separated by addition of dextran-coated charcoal and centrifugation at 2100×g for 15 minutes at 4 C. Supernatants from GR assays are decanted and counted in a Beckman LS-1800 liquid scintillation counter. Supernatants containing PR are pipetted into 24-well microplates and counted in a Packard TopCount liquid scintillation counter. Counts per minute (cpm) are entered into Packard's RIASMART for calculation of EC₅₀'s. Relative binding affinity for each test compound is calculated as follows: (EC₅₀ of standard)/(EC₅₀ of competitor)×100. The standard for the PR binding assays is P4 and the standard for the GR binding assays is dex.

### EXAMPLE 2

### Measuring Antiglucocorticoid Activity and Progesterone Antagonist Activity in vivo

For measuring in vivo progesterone antagonist activity of test compounds, T47D-CO human breast cancer cells, grown in monolayer culture in phenol red-free DMEM supplemented with 10% fetal bovine serum (FBS), 10 U/ml penicillin G and 10 μg/ml streptomycin sulfate, are transfected with a suitable hormone sensitive reporter gene plasmid, for example PRE₂-tk-LUC, which contains two copies of a progestin/glucocorticoid/androgen response element upstream of the thymidine kinase (tk) promoter and the firefly luciferase (LUC) reporter gene. Transfected T47D-CO cells are incubated with a (predetermined) maximum stimulatory concentration of a progestogen, for example P₄, in the absence or presence of various concentrations of test compound for 20 hours. LUC activity is determined using Promega's Luciferase Assay System and the IC₅₀ of the test compound is determined.

For measuring in vivo glucocorticoid antagonist activity, HepG2 human hepatoblastoma cells, grown in monolayer culture in phenol red-free MEMα supplemented with 10% FBS and pen/strep, are cotransfected with a suitable hormone sensitive reporter gene plasmid such as PRE₂-tk-LUC and a GR expression plasmid. Transfected HepG2 cells are incubated with a (predetermined) maximum stimulatory concentration of dexamethasone in the absence or presence of various concentrations of test compound for 20 hours. IC₅₀ of the test compound is determined by measuring LUC activity.

### EXAMPLE 3

### Chronic Daily Administration of CDB-4124 is Associated with Toxic Liver Effects

Initial studies conducted with Proellex (aka CDB-4124) demonstrated efficacy of the drug at every dose tested. Development of Proellex has focused on the two highest doses tested, 25 mg and 50 mg based on data suggesting that higher doses suppressed endometrial thickening and the potential for breakthrough uterine bleeding. Neither animal preclinical studies nor small trials in women in Europe at the higher doses for periods of up to six months of exposure predicted the liver toxicity exhibited in the Phase III clinical studies conducted in a diverse population in the United States. Proellex, delivered orally at a dose of 50 mg/day, exhibited severe liver toxicity in roughly 3-4% of the women receiving this dose. At 12.5 mg there were no adverse liver toxicity signals different from placebo. The maximum concentrations of CDB-4124 and its mono-demethylated metabolite (CDB-4453) for the 12.5 mg dose were 25% of the 50 mg dose. All liver toxicities resolved in those women that returned for safety follow-ups, including those subjects that developed liver-associated serious adverse effects (SAEs). The effects observed when Proellex was administered orally at 50 mg/day were significantly lower in frequency and intensity when Proellex was delivered at 25 mg/day. This observation was further amplified by the fact that longer durations of exposure have been safely achieved at a 25 mg/day dose than at a 50 mg/day dose suggesting that duration of exposure at lower doses does not necessarily result in the same liver toxicity than that observed at the 50 mg/day dose.

To date, over 600 patients, including women with confirmed cases of endometriosis or uterine fibroids, have participated in double blind and open label clinical trials in which patients were administered daily oral capsules containing doses of 12.5 mg, 25 mg or 50 mg CDB-4124 (Proellex) for over one month. Of these patients, about 500 received Proellex and about 130 received a placebo. Of the patients receiving Proellex, about 190 received a dose of 50 mg CDB-4124 per day, about 260 received a dose of 25 mg CDB-4124 per day and about 55 received a dose of 12.5 mg per day.

Liver enzymes were frequently monitored in participating subjects. The liver enzyme level at which the clinical trials would be discontinued was set at an increase in liver aminotransferases greater than, or equal to three times the Upper Limit of Normal (≧3×ULN).

During clinical trials, thirteen subjects were found to exhibit an increase in liver enzymes≧3×ULN, but this was confirmed by a repeat test in 48 hours in only nine subjects. Of the nine subjects with a confirmed increase in liver enzymes≧3×ULN, seven were severe enough elevations to be reported to the FDA as SAEs. One of these seven subjects had been receiving a dose of 25 mg CDB-4124 per day; the remaining six subjects had been receiving a dose of 50 mg CDB-4124 per day. Liver enzymes≧3×ULN persisted in five of the nine subjects with a confirmed increase in liver enzymes≧3×ULN. These five subjects had previously been dosed with the 50 mg dose. One of these subjects is receiving oral medication for treatment of her liver condition. Clinical trials involving CDB-4124 at all doses were voluntarily suspended as a result of these SAEs and were subsequently placed on clinical hold by the United States Food and Drug Administration for safety reasons.

Pharmacokinetic studies performed on participating subjects detected a high Cₘₐₓ and a Tₘₐₓ at 1-2 hours following administration. Large quantities of the monodemethylated metabolite of CDB-4124 were also detected, clearly indicating first pass metabolism of the antiprogestin. Providing further evidence of first pass metabolism, primary cultures of human and animal hepatocytes rapidly produce the mono-demethylated metabolite of CDB-4124. Metabolism of CDB-4124 by the liver provides the opportunity for liver damage and greatly reduces the concentration of the antiprogestin before it reaches the systemic circulation. Thus, alternative routes of administration of antiprogestins that avoid first pass metabolism such as, without limitation, intravenous, intramuscular, and sublingual, should allow antiprogestins to be absorbed directly into the systemic circulation and thereby provide a method for treating progestone-dependent conditions while avoiding liver toxicity. Administration routes which avoid first pass metabolism may also require less drug per dose to achieve the same therapeutic benefit relative to oral administration.

Pre-clinical studies were performed on rodents with breast tumors induced by 7,12-Dimethylbenz(a)anthracene (DMBA). These studies demonstrated efficacy of non-oral delivery methods of CDB-4124. In particular, CDB-4124 delivered by subcutaneous injection was effective in reducing the quantity and size of DMBA-induced breast tumors providing proof of concept.

### EXAMPLE 4

### Vaginal Delivery of CDB-4124 and CDB-4453 Reduces Systemic Concentrations Compared to Oral Administration and Avoids First Pass Metabolism

Beagles were administered 25 mg of CDB-4124 or CDB-4453 (the monodemethylated metabolite of CDB-4124) formulated as either a micronized powder or a vaginal suppository. As illustrated at FIG. 1, CDB-4124 and CDB-4453, when administered orally as a micronized powder, are rapidly metabolized after a peak plasma concentration (Cmax) is achieved. In contrast, when the same compounds are administered locally via vaginal suppository, the drugs are metabolized slowly and peak plasma concentrations (Cmax) are relatively low. Moreover, systemic exposure of the drug is much lower when administered locally (compare AUC for CDB-4124 and CDB-4453 when administered vaginally vs. orally).

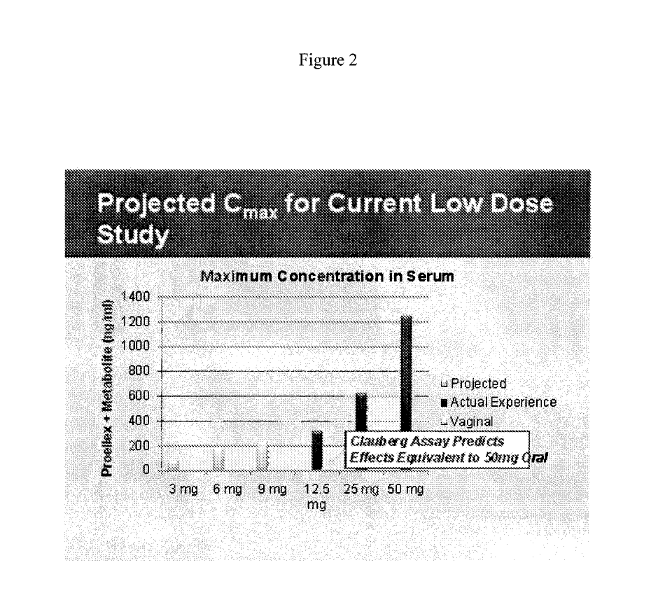

The maximum circulating concentrations (Cmax) of CDB-4124 obtained following vaginal administration to beagles were extrapolated to humans for the 12.5 mg, 25 mg and 50 mg doses actually administered during the Phase III clinical studies. As can be seen from FIG. 2, the predicted Cmax for vaginal administration of the 12.5 mg dose of CDB-4124 in humans is approximately 6.5% of the same dose when administered orally and the predicted Cmax for vaginal administration of the 50 mg dose of CDB-4124 in humans is approximately 2% of the same dose when administered orally.

### EXAMPLE 5

### Bioavailability of CDB-4124 at the Uterus is Surprisingly Low when Administered Orally

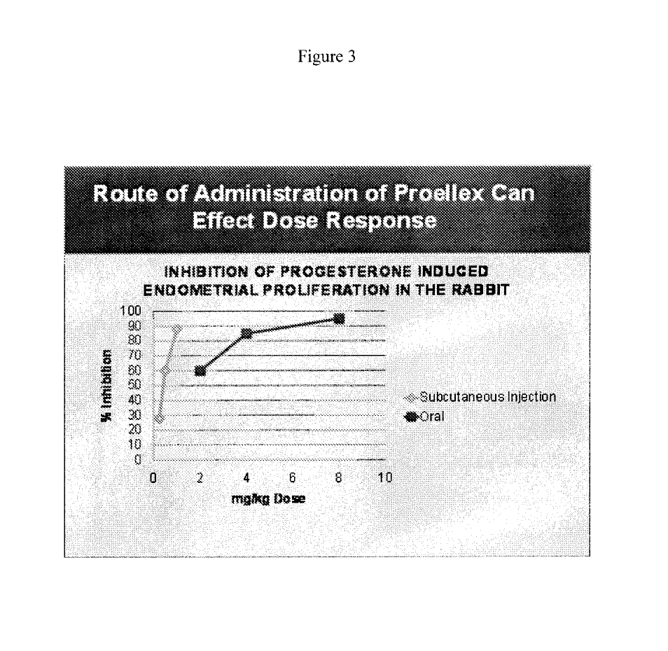

To determine whether the low circulating levels of CDB-4124 when administered locally could have any impact predictive of efficacy, an anti-Clauberg study was run in which immature estradiol-primed rabbits were coadministered progesterone and various doses of CDB-4124 by either subcutaneous or oral administration. At least 3 different highly trained individuals evaluated the rabbit uterus for glandular growth, for complexity and overall progesterone-induced “development”. The inhibition (by percentage) of progesterone-induced endometrial proliferation at each dose was assayed. As illustrated at FIG. 3, maximal inhibition was observed at a dose of less than 1 mg/kg when CDB-4124 was administered subcutaneously. However, maximal inhibition required a ˜8-fold increase in dosage when administered orally (i.e. 8 mg/kg). Importantly 8 mg/kg corresponds closely to the 50 mg/day dose of CDB-4124 administered to the female subjects described in Example 3. This demonstrates that the effective local concentration of CDB-4124 at the endometrium is greatly decreased when the drug is administered orally, most likely due to first-pass metabolism of the drug. Accordingly, in order to achieve therapeutic effect, e.g. for indications localized to the pelvic and reproductive tract, a relatively high dosage of CDB-4124 is required when administered orally, corresponding closely to the dosage of CDB-4124 at which toxic liver effects were observed in Example 3.

Another anti-Clauberg study was run in which immature estradiol-primed rabbits were administered progesterone alone (vehicle control) or were co-administered progesterone and three doses of CDB-4124 by either vaginal or oral administration. The inhibition of progesterone-induced endometrial proliferation at each dose was assayed. FIG. 3 illustrates the decrease in the McPhail index following increasing doses of CDB-4124 administered by either route. Maximal inhibition (i.e. a decrease in the McPhail index to 1.5) occurred at 0.2 mg/kg CDB-4124 when administered vaginally, compared to 0.8 mg/kg when administered orally. The data from this study show that vaginal delivery of CDB-4124 exhibits four times the antiprogestational activity of the same oral dose.

Cumulatively, the data indicate that a four-fold lower dose of antiprogestin can be administered vaginally compared to the effective dose when orally administered, while attaining only a small fraction of the maximal circulating concentrations compared to oral administration, thereby avoiding liver toxicity. For example, equivalent antiprogestational activity at the uterus is observed for a 50 mg oral dose of CDB-4124 and a 12.5 mg vaginal dose; however, the Cmax observed with a 12.5 mg vaginal dose is only 2% that observed with a 50 mg oral dose. The relatively high local concentration of the drug achieved by local administration allows a relatively low dose of the drug (compared with oral administration) to achieve therapeutic effect for indications localized to the pelvic and reproductive tract (e.g. endometriosis, uterine fibroids and ovarian cancer). Because a high concentration of the drug in the systemic circulation (and associated first pass metabolism of the drug) is not reached by local administration, avoidance of the severe liver toxicity observed in a small percentage of subjects following oral administration of CDB-4124 in previous Phase III clinical studies at doses of 25 and 50 mg is a surprising advantage of administering the drug locally. Similar advantages should inure to local administration of other antiprogestins.

### EXAMPLE 6

### Effect of R¹ and R² Substituents on Thermodynamic Solubility

Thermodynamic solubility of the following compounds were tested and compared to that of CDB-4124:

21-methoxy-17α-acetoxy-11β-(4-acetylphenyl)-19-norpregna-4,9-diene-3,20-dione (in which R¹ is at the para position and is —COCH₃, R² is methoxy, R³ is acetoxy, R⁴ is methyl, and X is ═O) (CDB-4239) with the following structural formula:

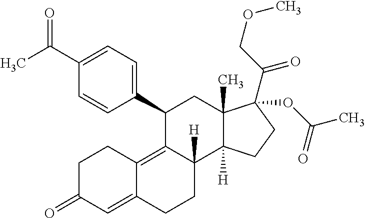

17α-acetoxy-11β-(4-acetylphenyl)-19-norpregna-4,9-dien-3,20-dione (in which R¹ is at the para position and is —COCH₃, R² is hydrogen, R³ is acetoxy, R⁴ is methyl, and X is ═O) (REP-4510) with the following structural formula:

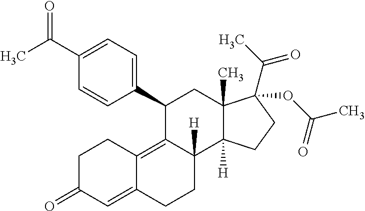

and 21-acetoxy-17α-acetoxy-11β-(4-acetylphenyl)-19-norpregna-4,9-diene-3,20-dione (in which R¹ is at the para position and is —COCH₃, R² and R³ are acetoxy, R⁴ is methyl, and X is ═O) (CDB-4241) with the following structural formula:

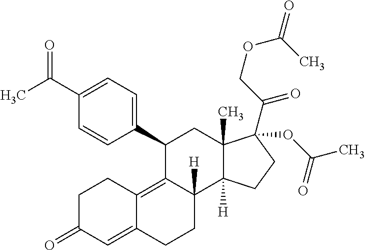

CDB-4239, CDB-4241 and REP-4510 were determined to be crystalline solids with the following characteristics:

| | DSC | | | TG |
|---|---|---|---|---|
| | | Peak | | weight loss |
| | Onset temp | maximum | | (%) up to |
| Compound | (° C.) | (° C.) | Enthalpy (J/g) | 120° C. |
| CDB-4239 | 105.5 | 110.1 | 7.9 | 1.0 |
| CDB-4241 | 208.2 | 209.5 | 55.3 | 0.1 |
| REP-4510 | 261.5 | 262.9 | 62.9 | 0.1 |

Briefly, 300 ul of solvent (ethanol, 0.1 M HCl or distilled H₂O) were added to 14-16 mg of the solid compound (5 parallel measurements per sample, per solvent, mean concentrations 93-107 mM). The mixtures were shaken at 37° C. for 24 and 72 hours. Concentrations of the filtrates were determined by liquid chromatography with UV detector (LC/UV) with 3-point calibration. No significant degradation was observed at the end of the measurements. Results are provided in Tables 1 and 2:

TABLE 1

| | 24 h | | | | | | |
|---|---|---|---|---|---|---|---|
| | Ethanol | | | 0.1M HCl | | Distilled water | |
| Compound | Mean | | SD | Mean | SD | Mean | SD |
| REP-4510 | 3.30 | mM | 0.08 | 0.50 μM | 0.03 | 0.52 μM | 0.04 |
| CDB-4241 | 26.27 | mM | 2.05 | 3.21 μM | 0.12 | 3.08 μM | 0.22 |
| CDB-4239 | >98.15 | mM* | — | 140.40 μM | 2.30 | 140.60 μM | 0.90 |
| CDB-4124 | 36.80 | mM | 6.24 | 53.09 μM | 1.92 | 7.10 μM | 0.50 |

*compound totally dissolved

TABLE 2

| | 72 h | | | | | | |
|---|---|---|---|---|---|---|---|
| | Ethanol | | | 0.1M HCl | | distilled water | |
| Compound | Mean | | SD | Mean | SD | Mean | SD |
| REP-4510 | 2.90 | mM | 0.09 | 0.45 μM | 0.01 | 0.44 μM | 0.02 |
| CDB-4241 | 21.58 | mM | 2.76 | 3.90 μM | 0.16 | 3.05 μM | 0.14 |
| CDB-4239 | >100.10 | mM* | — | 110.20 μM | 11.80 | 54.80 μM | 4.20 |
| CDB-4124 | 34.10 | mM | 5.99 | 56.12 μM | 0.73 | 3.20 μM | 0.60 |

*compound totally dissolved

Crystal form was checked by X-ray powder diffraction (XRPD) after recovering the slurry at 24 and 72 hours. No form change was observed with any of the investigated compounds during suspension slurring. A decreasing tendency of the water and 1.0 M HCl solubilities of CDB-4239 measured at 24 and 72 hours was observed. This could not be rationalized by changing to a more stable form. The exact cause of this observed phenomenon is not known.

Analysis of these data indicates that compounds of general formula I having R¹=acyl (particularly COCH₃), alkylsulfinyl or alkylsulfonyl at the meta (or ortho) position and having R²=alkoxy, particularly methoxy, will be surprisingly soluble and will retain antiprogestational activity with low antiglucocorticoid activity.

What is claimed is:

1. A compound of the general formula: 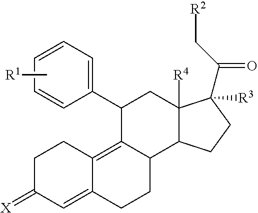
or a pharmaceutically acceptable salt thereof wherein: R¹ is at the para position and is —SO₂CH₃ or —SOCH₃; R² is selected from the group consisting of: hydrogen, alkoxy and acyloxy; R³ is selected from the group consisting of: alkyl, hydroxy, alkoxy, and acyloxy; R⁴ is methyl; and X is ═O.

2. A compound or salt thereof in accordance with claim 1 wherein R¹ is —SO₂CH₃.

3. A compound or salt thereof in accordance with claim 2 wherein R² is hydrogen.

4. A compound or salt in accordance with claim 3 wherein R³ is acetoxy.

5. A compound or salt thereof in accordance with claim 1 wherein R¹ is —SOCH₃.

6. A compound or salt thereof in accordance with claim 5 wherein R² is hydrogen.

7. A compound or salt thereof in accordance with claim 6 wherein R³ is acetoxy.

8. A compound or salt thereof in accordance with claim 1 wherein R¹ is —SO₂CH₃; R² is methoxy; and R³ is acetoxy.

9. A compound or salt thereof in accordance with claim 1 wherein R¹ is —SOCH₃; R² is methoxy; and R³ is acetoxy.

10. A pharmaceutical composition comprising a therapeutically effective amount of a compound or salt thereof according claim 1 and a pharmaceutically acceptable excipient.

11. A method for producing an antiprogestational effect in a patient, comprising administering to said patient a therapeutically effective amount of a compound or salt thereof in accordance with claim 1.

12. A method for treating a progesterone-dependent condition selected from the group consisting of endometriosis and pain associated therewith, adenomyosis, endometriomas of the ovary, dysmenorrhea, uterine fibroids, endometrial hyperproliferation, ovarian cancer, and cervical cancer comprising administering to a patient in need thereof a therapeutically effective amount of a compound or salt thereof in accordance with claim 1.

13. A method for treating a progesterone-dependent condition selected from the group consisting of endometriosis and pain associated therewith, adenomyosis, endometriomas of the ovary, dysmenorrhea, uterine fibroids, endometrial hyperproliferation, ovarian cancer, and cervical cancer comprising administering to a patient in need thereof a composition in accordance with claim 10.

14. The method of claim 13 wherein the composition is administered via a route selected from the group consisting of: oral, vaginal, intrauterine and topical.

15. The method of claim 14 wherein the composition is administered to the vaginal mucosa of the patient.

16. The method of claim 12 wherein the compound is administered daily.

17. The method of claim 16 wherein the compound is administered at a dosage of 2 mg to 80 mg per day.
